(19) United States
(12) Patent Application Publication
(10) Pub. No.: US 2018/0051241 A1
(43) Pub. Date: Feb. 22, 2018

(54) Cultivation Bottle for Mycelium of Inonotus Obliquus

(71) Applicant: Timing Pharmaceutical Co., LTD, New Taipei City (TW)

(72) Inventor: Po-Lun Wang, New Taipei City (TW)

(21) Appl. No.: 15/402,226

(22) Filed: Jan. 9, 2017

(30) Foreign Application Priority Data
Aug. 17, 2016 (TW) 105120377

(51) Int. Cl. C12M 1/24; C12M 1/00; C12M 1/12; C12N 1/14

(52) U.S. Cl. CPC C12M 23/08; C12N 1/14; C12M 25/06; C12M 23/38

(57) ABSTRACT

A cultivation bottle for mycelium of Inonotus obliquus is provided. The cultivation bottle includes a bottle having a hollow shape and configuring to contain a matrix which includes an agar plate, sawdust distributed uniformly in the agar plate, and Inonotus obliquus strain seeded in the agar plate; and a bottle cap configured to cover the opening of the bottle. The bottle cap includes an annular cap defining a space, a press cap disposed in the space and having a bottom part and a press part; and at least one elastic member disposed under the press part within the space, so that the press part and the bottom part enclose the space for containing culture liquid.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present disclose is related to a cultivation bottle, more particularly, to a cultivation bottle for mycelium of Inonotus obliquus.

### Description of Related Arts

Inonotus obliquus is a member under Kingdom Fungi, Division Basidiomycota, Class Agaricomycetes, Order Hymenochaetales, Family Hymenochaetaceae, Genus Inonotus, and has aliases such as Chaga mushroom, birch mushroom, Inonotus Bacteria, Siberia Ganoderma, birch hole mushroom, and tree mushroom. Inonotus obliquus is one kind of folk medicinal mushroom which locally abundant in Russia and Eastern Europe. According to the research, Inonotus obliquus has effects of preventing adenocarcinoma, hepatoma, uterine cancer, stomach cancer, diabetes and hypertension. In recent years, it has caused extensive concern on the nutritive and medicinal value of Inonotus obliquus, and Inonotus obliquus becomes a popular field in pharmaceutical industry and health foods industry.

In recent years, more and more researches show that Inonotus obliquus has amount of plant cellulose polysaccharide, and the materials of polysaccharide, such as glucans, isopolysaccharides and pectins, may further provide one of ingredients of the cell membrane. In particular, the biological response modifiers (BRM) materials of the polysaccharide have a high content of glucan which has effect of promoting the immune cell activity, suppressing the spread of cancer cell, preventing recurrent cancer, and preventing the absorption of carcinogen in stomach.

Solid culture can be operated in a low moisture content condition and provide a selectivity growth environment for mycelium, so it is suitable for fungi, especially cultivation as an edible and medicinal mushroom. The fungi is not in liquid place in native, and mycelium grown in the liquid culture and the native growing has different metabolic pathways, so also has different medicinal properties naturally. For Inonotus obliquus, the solid culture is more similar to native environment than the liquid culture, so products and nutritional ingredients of Inonotus obliquus grown in the solid culture are also similar to that of native Inonotus obliquus.

Since the rare success of traditional artificial culture, high cost of bacteria strain, and easy microbial contamination or disease in early growth stage, a container with high tightness is usually used for culture to prevent from contamination, but it causes problems of high temperature, poor gas flow, lack of oxygen within the container, and a high concentration of metabolic materials in a late growth stage, which results in non-germination, growth retardation, less competition to microorganism and low nutritional ingredients of metabolic products. In addition, the traditional culture has a complicated process and a lot of labor costs on the management of the process, such as observation and control of the input of culture liquid, the ventilation of device, and the temperature and moisture of environment. If the environmental condition is controlled unsuitable for nurturing, the production state and product quality of mycelium of Inonotus obliquus may be affected, and the yield of production is reduced.

Therefore, what is need is to develop a cultivation bottle capable of facilitating control of the environment condition for nurturing, so as to maintain the production state and product quality of mycelium of Inonotus obliquus.

### SUMMARY OF THE PRESENT INVENTION

An objective of the present disclosure is to provide a cultivation bottle for the mycelium of Inonotus obliquus. The cultivation bottle contains a matrix with an agar plate where Inonotus obliquus is seeded, so that it is easy to control the environmental condition for nurturing, and maintain the production state and product quality of mycelium of Inonotus obliquus.

Another object of the present disclosure is to provide a cultivation bottle for mycelium of Inonotus obliquus, to enable the operator to release the culture liquid into the matrix with agar plate where Inonotus obliquus is seeded by an elastic press operation, so as to provide a culture environment for nurturing with well-controlled ventilation, temperature and moisture, and to maintain the production state and product quality of mycelium of Inonotus obliquus.

To achieve the foregoing objective, the present disclosure provides a cultivation bottle for mycelium of Inonotus obliquus. The cultivation bottle includes a bottle having a hollow shape with an opening; and a bottle cap configured to cover the opening of the bottle. The bottle cap includes an annular cap defining a space formed inside; a press cap having a bottom part and a press part, and disposed in the space; and at least one elastic member disposed under the press part and in the space, to enable the press part and the bottom part to enclose the space.

Preferably, the annular cap includes an annular part having an inner periphery and a plurality of ventilation holes cut therethrough; a rotatable part disposed under the annular part and configured to rotate to enclose a part or all of the ventilation holes; and an annular wall extended from the inner periphery of the annular part into the bottle and configured to define the space. The annular wall has a structure formed on a lower periphery thereof and gradually-extended outwardly, the bottom part of the press cap has a soft rubber disposed on a periphery edge thereof, so as to prevent the culture liquid within the space from leaking when the press part is not pressed.

Preferably, the press cap has a rod disposed between the press part and the bottom part, and when the press part is not pressed, the elastic member is compressed to enable the bottom part to abut against the lower edge of the annular cap; and while the press part is pressed, the rod presses the bottom part to form a slit between the bottom part and a lower periphery of the annular cap, so that the culture liquid can be released into the matrix.

According to the cultivation bottle for mycelium of Inonotus obliquus of the present disclosure, the operator may elastically press the cultivation bottle to easily release the culture liquid into the matrix having the agar plate where Inonotus obliquus is seeded, and to provide an culture environment for nurturing with well-controlled ventilation, temperature and moisture, and to maintain the production state and product quality of mycelium of Inonotus obliquus. In addition, the cultivation bottle of the present disclosure is capable of improving the germination and growth of mycelium of Inonotus obliquus, and providing the rotatable manner to quickly control the air flow to make the temperature and moisture in the cultivation bottle equal to that of indoor environment, so that the nurturing process may become simpler, prevent the potential contamination when the bottle cap is opened, and have an effect of reducing the cost of labors and equipment. The cultivation bottle of this present disclosure is operated to input the culture liquid by the press manner, so it is quick and convenient to control the germination time and growth rate. When the mycelium grows into a certain degree of size, the operator can harvest and extract the mycelium without replacing the bottle or seeding in twice for subculture, so that the nurturing process may be simpler and faster.

### BRIEF DESCRIPTION OF THE DRAWINGS

The detailed structure, operating principle and effects of the present disclosure will now be described in more details hereinafter with reference to the accompanying drawings that show various embodiments of the present disclosure as follows.

FIG. 1 is a schematic view of a first embodiment of an incubation bottle for mycelium of Inonotus obliquus of the present disclosure, illustrating that the bottle cap is not pressed yet.

FIG. 2 is an exploded view of a bottle cap of the first embodiment of the present disclosure.

FIG. 3 is a schematic view of the first embodiment of the incubation bottle for mycelium of Inonotus obliquus of the present disclosure, illustrating that the bottle cap is kept being pressed to drop the culture liquid on the matrix within the bottle.

FIG. 4 is a schematic view of the first embodiment of the incubation bottle for mycelium of Inonotus obliquus, illustrating that mycelium of Inonotus obliquus is incubated in the matrix within the bottle.

FIG. 5 is a schematic view of a second embodiment of the incubation bottle for mycelium of Inonotus obliquus of the present disclosure, illustrating that the bottle cap is not pressed yet.

FIG. 6 is a cross-sectional view of the bottle cap with a rotatable part of the present disclosure, illustrating that the rotatable part is able to enclose a part or all of ventilation holes.

FIGS. 7A, 7B and 7C are top plain views of the rotatable part of the bottle cap shown in FIG. 6, respectively illustrating that the rotatable part does not enclose the ventilation holes, encloses a part and all of the ventilation holes.

FIGS. 8A and 8B are schematic views of a third embodiment of the incubation bottle for mycelium of Inonotus obliquus of the present disclosure, respectively illustrating that the bottle cap is not pressed yet, and is pressed already.

FIGS. 9A and 9B are schematic views of a fourth embodiment of the incubation bottle for mycelium of Inonotus obliquus of the present disclosure, respectively illustrating that the bottle cap is not pressed yet, and pressed already.

### DETAILED DESCRIPTION OF THE PREFERRED EMBODIMENT

The technical content of the present invention will become apparent by the detailed description of the following embodiments and the illustration of related drawings as follows.

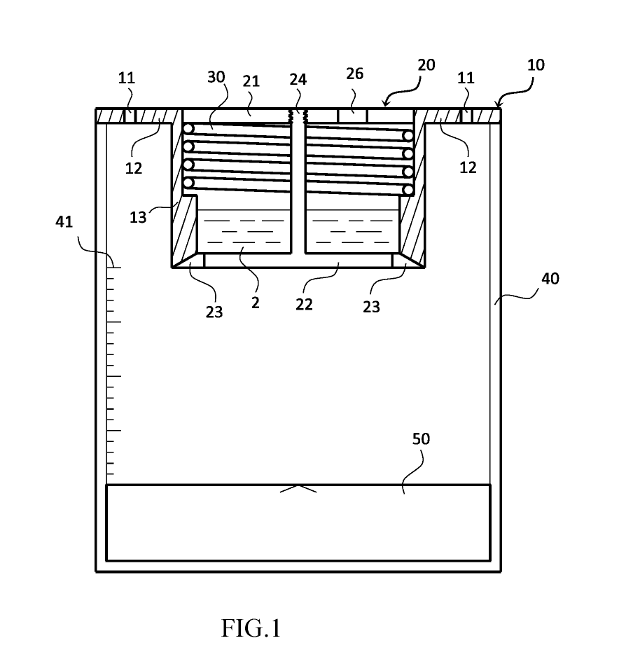

Please refer to FIG. 1 which shows a schematic view of a first embodiment of an incubation bottle for mycelium of Inonotus obliquus of the present disclosure. In the first embodiment, the incubation bottle mainly includes a bottle 40 and a bottle cap. The bottle 40 is in a hollow shape and has an opening covered by the bottle cap. The bottle 40 contains a matrix 50 which includes an agar plate, sawdust distributed uniformly in the agar plate, and Inonotus obliquus strain seeded into the agar plate. The matrix 50 can be a solid medium matrix mainly including the agar plate. To manufacture the matrix 50, the agar is doped and uniformly mixed with some nutrients and sawdust of birch or other species of tree, for germination and growth of Inonotus obliquus strain.

For example, the bottle 40 can be a transparent and cylindrical bottle with an about 10˜20 cm of diameter and a 15˜30 cm of height. The bottle 40 includes a tight member (such as rubber) disposed on an edge of the opening, so that the bottle 40 can form an enclosed space when the opening is covered by the bottle cap. The enclosed space is used to accommodate culture liquid.

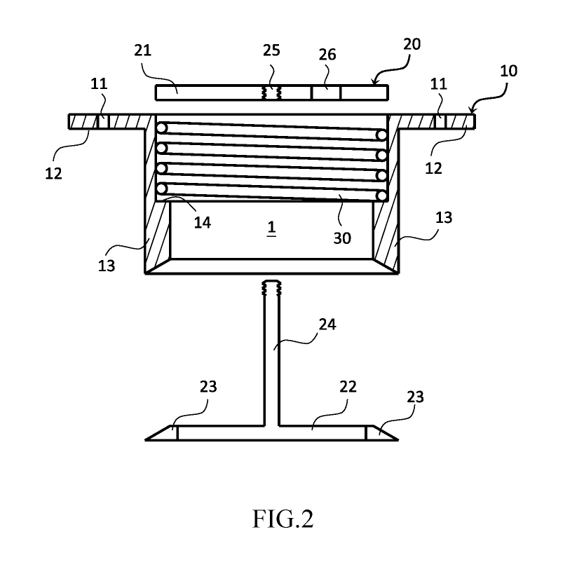

Please also refer to FIG. 2 which shows an exploded view of the first embodiment of the bottle cap of the present disclosure. The bottle cap includes an annular cap 10, a press cap 20 and an elastic member 30. The annular cap 10 includes an annular part 12 and an annular wall 13, and the annular part 12 is configured to cover the opening of the bottle 40 by an outer edge thereof, and the annular wall 13 is downwardly extended from an inner edge of the annular part 12 into the bottle 40. The annular wall 13 defines space 1. The annular part 12 is formed with a plurality of ventilation holes 11 cut therethrough. The press cap 20 includes a press part 21 and a bottom part 22. The bottom part 22 is in a circular shape and has a soft rubber 23 disposed on a periphery thereof. The bottom part 22 is used to seal a lower edge of the annular wall 13, and the lower edge of the annular wall 13 has a gradually-extended structure which is extended from inside to outside, so as to seal with the soft rubber 23 well. The bottom part 22 has a rod 24 formed at a central portion thereof, and the rod 24 has a top with a thread to be screwed into a thread hole 25 formed at a central portion of the press part 21. The elastic member 30 is disposed in the space 1 and under the press part 21, the annular wall 13 has a step structure 14 formed on an inner side surface thereof to support the elastic member 30. Preferably, the elastic member 30 can be a helical spring.

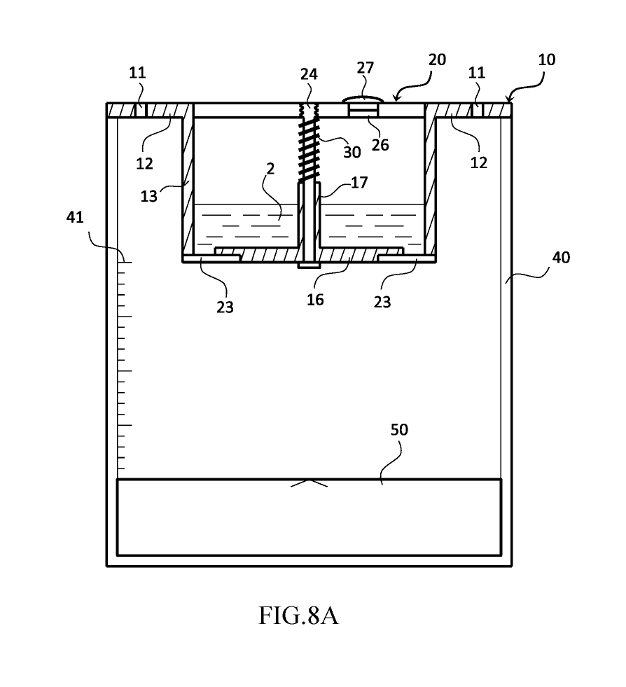

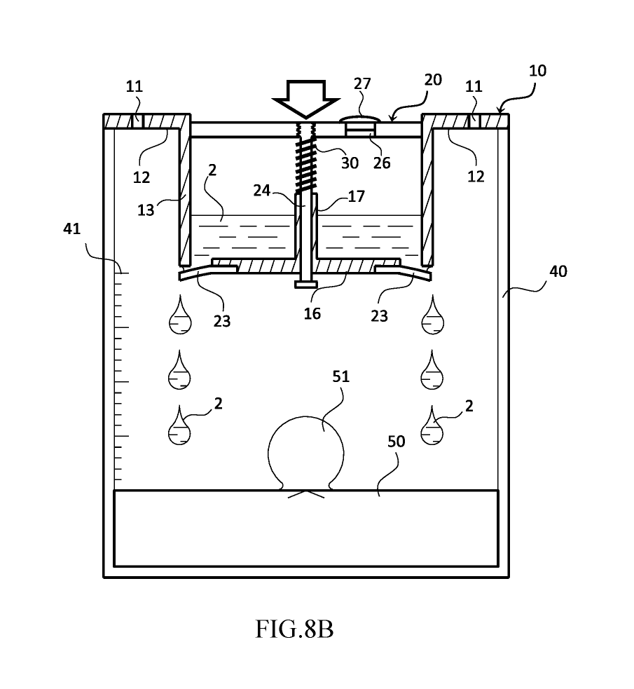

To assemble the bottle cap of the first embodiment of the present disclosure, the bottom part 22 is inserted into the space 1 through a bottom side of the space 1, the elastic member 30 is placed on the step structure 14 in the space 1, and the press part 21 is screwed and locked with the top of the rod 24 of the bottom part 22, so that the elastic member 30 is compressed in the space 1 and supported by the step structure 14 to press against a lower surface of the press part 21, thereby providing an upward elastic force to the press part 21. As a result, the soft rubber 23 on the periphery of the bottom part 22 can seal the lower edge of the annular wall 13, and the press part 21, the annular wall 13 and the bottom part 22 together define the enclosed space 1 to contain culture liquid 2. The culture liquid 2 is a solution mainly including nutrient content, such as carbohydrate. The press part 21 has a liquid inlet 26 cut therethrough and configured to input the culture liquid 2 into the space 1. The liquid inlet 26 can be enclosed by a sealing cover 27, as shown in FIG. 8A.

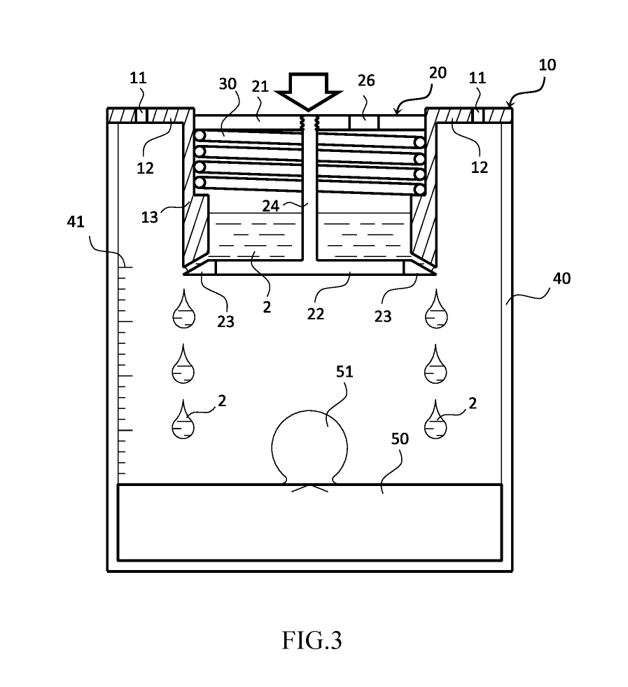

Please refer to FIG. 3 which shows a schematic view of the first embodiment of the incubation bottle for mycelium of Inonotus obliquus of the present disclosure. In FIG. 3, the bottle cap is pressed to leak the culture liquid 2 on the matrix 50 in the bottle. The press part 21 of the press cap 20 is locked with the rod 24 of the bottom part 22, so the press part 21 and the bottom part 22 can be acted simultaneously by the rod 24. When the press part 21 is pressed (in a direction of an arrow shown in FIG. 3), the rod 24 presses the bottom part 22 to form a slit between the soft rubber 23 and the lower periphery of the annular cap 13 of the annular cap 10, so that the culture liquid 2 can be dropped on the matrix 50 for incubation of the mycelium 51 of Inonotus obliquus. A flow of the culture liquid 2 can be adjusted upon demand. When the press part 21 is not pressed, the press part 21 and the bottom part 22 are moved back their original positions by the elastic force, so as to enable the soft rubber 23 of the bottom part 22 to tightly attach the lower edge of the annular wall 13 again, to prevent the culture fluid from flowing on the matrix.

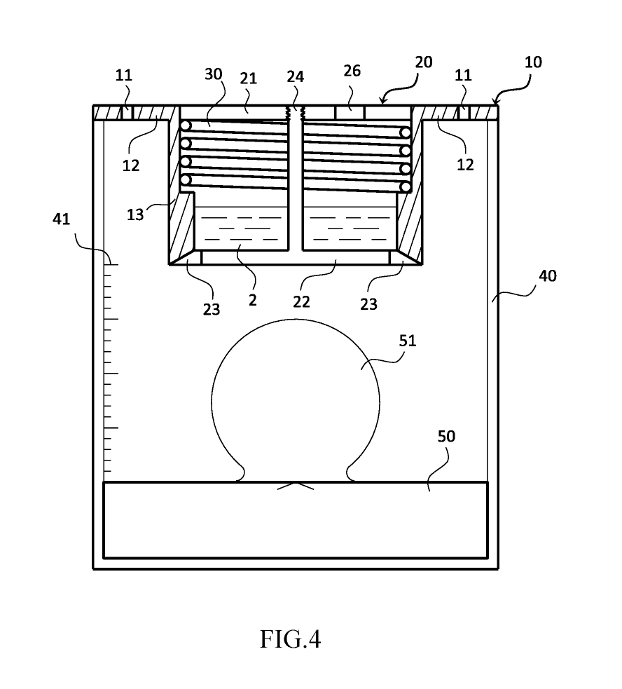

Please refer to FIG. 4 which shows a schematic view of the first embodiment of the incubation bottle of mycelium of Inonotus obliquus of the present disclosure. In FIG. 4, the mycelium of Inonotus obliquus 51 is cultivated on the matrix 50 in the bottle 40. The bottle 40 has a side wall with graduation 41 to easily observe a height of the mycelium of Inonotus obliquus 51 during germination and growth of mycelium of Inonotus obliquus 51. In the embodiment, a thickness of the matrix 50 is preferably from 5 cm to 10 cm, and the best time to harvest the mycelium of Inonotus obliquus 51 is when the mycelium of Inonotus obliquus 51 has a 7-10 cm of diameter, and the same time, the mycelium of Inonotus obliquus 51 has an about 5-8 cm of height and has better nutrient content.

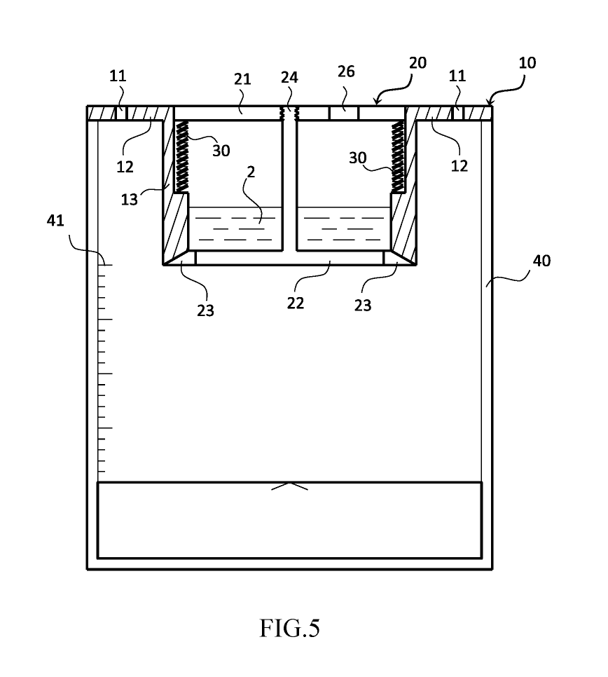

Please refer to FIG. 5 which shows a schematic view of a second embodiment of the incubation bottle for mycelium of Inonotus obliquus of the present disclosure. In FIG. 5, the bottle cap is not pressed yet. Compared with the first embodiment, the bottle cap of the second embodiment includes the annular cap 10, the press cap 20 and a plurality of elastic members 30. The annular cap 10 and the press cap 20 are the same as that of the first embodiment, so their detailed descriptions are omitted. The plurality of elastic members 30 are symmetrically distributed on the support structure of the annular wall 13 and pressed against the lower surface of the press part 21. The second embodiment of the incubation bottle for mycelium of Inonotus obliquus can achieve same effect and result as the first embodiment.

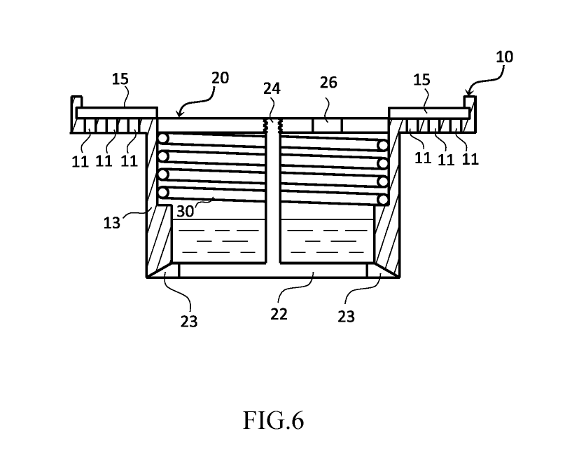

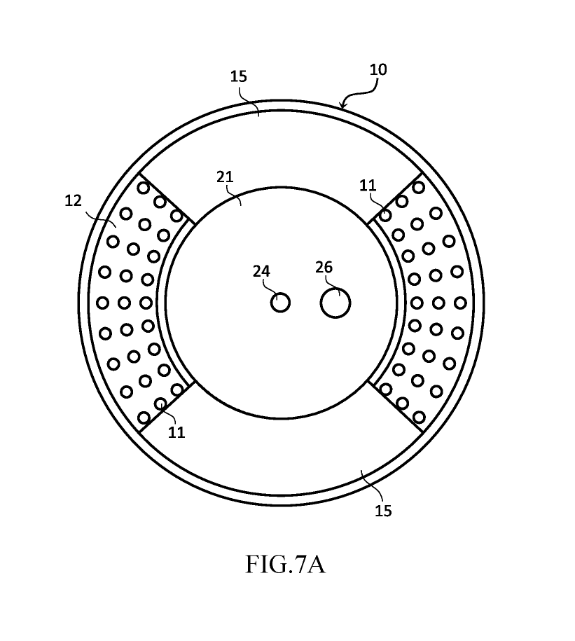

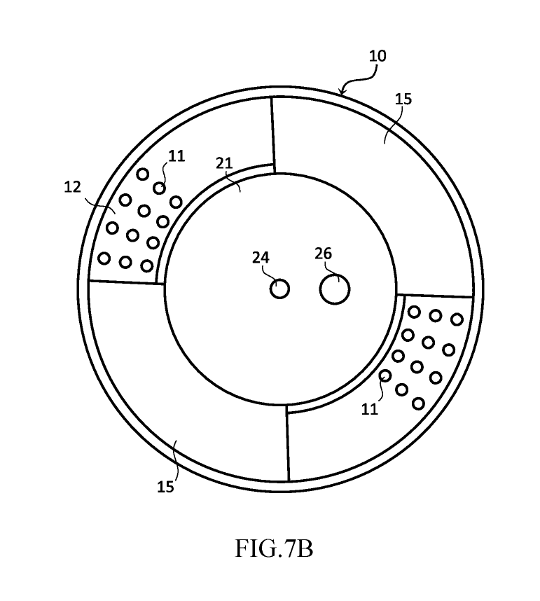

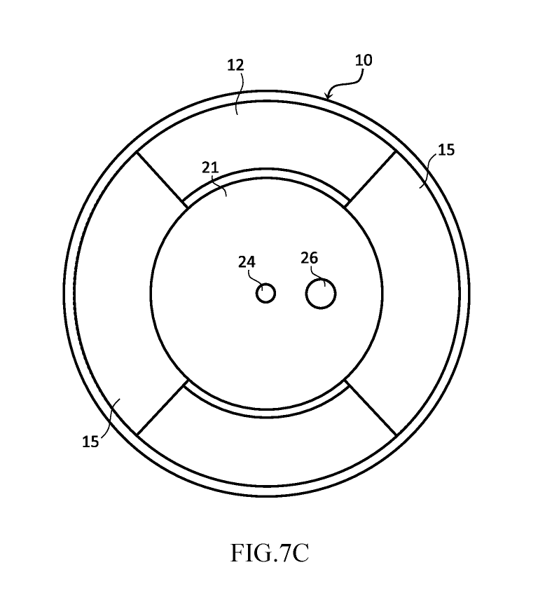

Please refer to FIG. 6 which shows a cross-sectional view of the bottle cap with a rotatable part of the present disclosure. In FIG. 6, the opening of the bottle 40 is coved by the bottle cap, similarly to FIG. 1. The bottle cap of FIG. 6 further includes a rotatable part 15 configured to enclose a part or all of ventilation holes 11, whereby the bottle 40 can has an enclosed space or a ventilative space inside. The rotatable part 15 is disposed over the annular part 12 of the annular cap 10, and the outer periphery of the annular part 12 is upwardly extended to form a mounting slot for mounting an outer periphery of the rotatable part 15. An operator can rotate the rotatable part 15 to perform circle movement along the mounting slot. The rotatable part 15 has a fan-shaped structure, as shown in FIG. 7A. While the rotatable part 15 is rotated to a different position, the rotatable part 15 does not enclose the ventilation holes 11, or encloses a part or all of the ventilation holes 11, respectively, as shown in FIGS. 7A, 7B and 7C.

Before in use, all embodiments of the incubation bottle for mycelium of Inonotus obliquus or combination thereof must be sterilized first, the matrix 50 is placed into the bottle after sterilization, and Inonotus obliquus strain is then seeded in the matrix 50. An adequate quantity of the culture liquid 2 is inputted into the space 1 via the liquid inlet 26. When the Inonotus obliquus strain is seeded in the matrix 50, germination of mycelium of Inonotus obliquus 51 will not happen because Inonotus obliquus requires fluid (such as water or culture liquid) for germination. The operator can rotate the rotatable part 15 to enclose all of the ventilation holes 11 of the bottle cap to prevent contamination caused by microorganism falling into the bottle. As a result, it is convenient to preserve Inonotus obliquus strain in the incubation bottle and control its growth time in batch production. The Inonotus obliquus strain can be preserved for about three weeks, and adding the culture liquid 2 is able to induce Inonotus obliquus strain to germinate mycelium of Inonotus obliquus 51. To start culturing mycelium of Inonotus obliquus 51, the operator can press the press part 21 to input adequate culture liquid 2, so as to induce germination of the mycelium of Inonotus obliquus 51. The adequate culture liquid 2 can further be inputted into bottle upon a growth state of individual mycelium of Inonotus obliquus 51, so as to control a growth speed and a size of the mycelium of Inonotus obliquus 51, and the time for harvesting mycelium of Inonotus obliquus 51.

In an early stage of germination of mycelium of Inonotus obliquus 51, lower oxygen exchange is required and production of carbon dioxide and heat is also less, and the bacteria infection must be prevented, so the rotatable part 15 of the annular cap 10 can be rotated to enclose the ventilation holes 11, as shown in FIGS. 6 and 7C, to facilitate germination of mycelium of Inonotus obliquus 51. After mycelium of Inonotus obliquus 51 grows to have an about 1-3 cm of diameter, the mycelium of Inonotus obliquus 51 requires more oxygen exchange and produces more carbon dioxide and heat, and microorganism only causes less damage, so the rotatable part 15 can be rotated to open the ventilation holes 11, as shown in FIG. 7A, to provide more gas exchange and hot gas dissipation for assisting growth of mycelium of Inonotus obliquus 51. In addition, the rotatable part 15 can be rotated to different location to adjust a degree of ventilation upon growth state of mycelium of Inonotus obliquus 51.

Please refer to FIGS. 8A and 8B which respectively show schematic views of a third embodiment of the incubation bottle for mycelium of Inonotus obliquus of the present disclosure. In FIGS. 8A and 8B, the bottle cap is not pressed yet, and kept being pressed, respectively. In the third embodiment of the present disclosure, the incubation bottle for mycelium of Inonotus obliquus mainly includes a bottle 40 in a hollow shape, and a bottle cap configured to cover an opening of the bottle 40. The bottle 40 contains a matrix 50 including an agar plate, sawdust uniformly distributed in the agar plate, and Inonotus obliquus strain seeded in the agar plate.

The bottle cap of the third embodiment includes the annular cap 10, the press cap 20 and the elastic member 30. The press cap 20 has the press part, and the annular cap 10 has the annular part 12, the annular wall 13 and a bottom part 16. The outer periphery of the annular part 12 is configured to cover the opening of the bottle 40, and the inner periphery of the annular part 12 is downwardly extended into the bottle 40 to form the annular wall 13. The bottom part 16 is formed by a lower periphery of the annular wall 13, and the annular wall 13 and the bottom part 16 define a space to contain the culture liquid 2. The bottom part 16 has a hollow cylinder 17 formed at a central portion thereof, and a rod 24 is inserted into the hollow cylinder 17 from down to up, and through the elastic member 30. The top of the rod 24 is locked with a central portion of the press cap 20. The bottom part 16 has a plurality of gaps which each is enclosed by a rubber diaphragm 23 to prevent leakage of the culture liquid 2.

As shown in FIG. 8B, when the press part of the press cap 20 is pressed (in a direction of an arrow of FIG. 8B), gas in the space is compressed first, and the compressed gas in the space forces the rubber diaphragm 23 to open the gaps of the bottom part 16, so that the culture liquid 2 in the space is dropped on the matrix 50. As a result, the third embodiment can achieve same effects and results as that of previous embodiments.

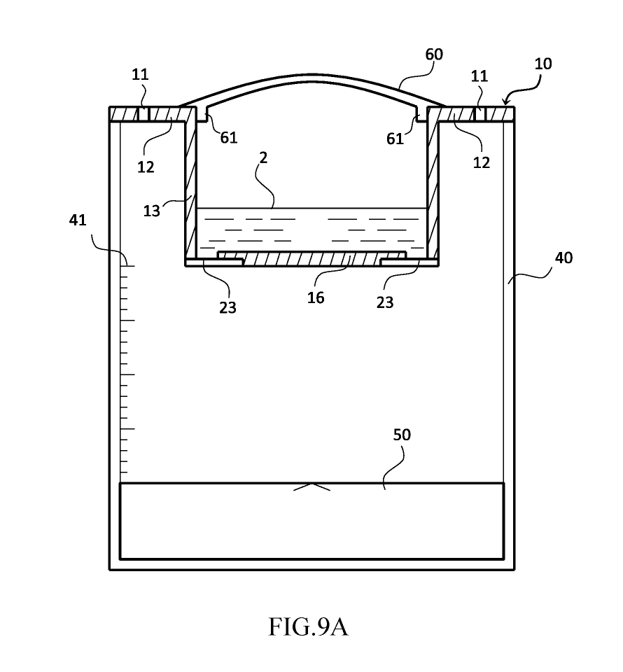

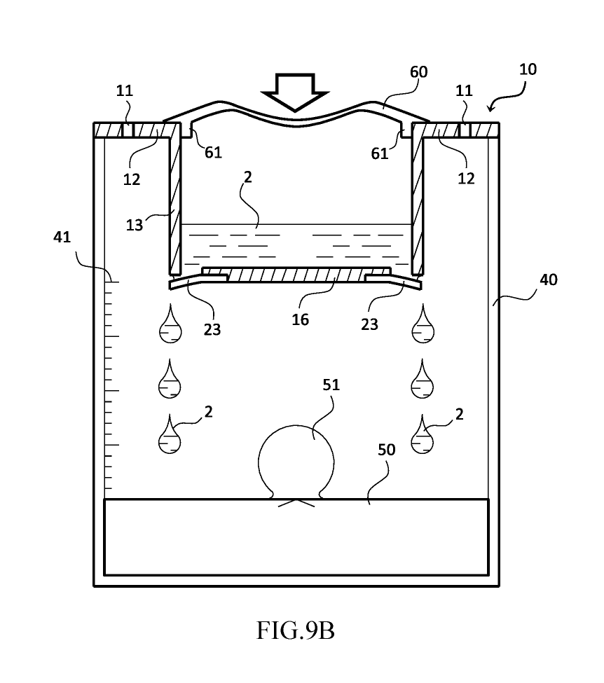

Please refer to FIGS. 9A and 9B which respectively show schematic views of a fourth embodiment of the incubation bottle for mycelium of Inonotus obliquus , in accordance with the present disclosure. In FIGS. 9A and 9B, the bottle cap is not pressed yet, and kept being pressed, respectively. In the fourth embodiment, the incubation bottle mainly includes a bottle 40 in a hollow shape and a bottle cap which is configured to cover an opening of the bottle 40. The bottle 40 contains the matrix 50 which includes the agar plate, sawdust uniformly distributed in the agar plate, and Inonotus obliquus strain seeded in the agar plate.

In the fourth embodiment, the bottle cap includes the annular cap 10 and a press cap 60. The annular cap 10 includes the annular part 12, the annular wall 13 and the bottom part 16, and the outer periphery of the annular part 12 is configured to cover the opening of the bottle 40, and the inner periphery of the annular part 12 is downwardly extended into the bottle 40 to form the annular wall 13. The bottom part 16 is formed by the lower edge of the annular wall 13, and the annular wall 13 and the bottom part 16 define the space to contain the culture liquid 2. The bottom part 16 has the plurality of gaps which each is enclosed by the rubber diaphragm 23 to prevent leakage of the culture liquid 2. In addition, the press cap 60 has a press part which is formed with an annular protrusion 61 disposed on a lower surface thereof. When the press cap 60 covers the annular cap 10, an outer periphery of the annular protrusion 61 matches and attaches with an inner periphery of the annular wall 13 to enclose the space, as shown in FIG. 9.

As shown in FIG. 9B, when the press part of the press cap 60 is pressed (as shown by the arrow), gas in the space is compressed and the compressed gas forces the rubber diaphragm 23 to open the gaps of the bottom part 16, so that the culture liquid 2 is dropped on the matrix 50. As a result, the fourth embodiment can achieve same effects and results as the previous embodiments.

The above description is for the purpose of illustration only and shall not be interpreted in any way to limit the scope, configuration or applicability of the present invention. A person skilled in the art may carry out many changes and modifications in the described embodiments without departing from the spirit and the scope of the present invention, which is intended to be limited only by the appended claims.

What is claimed is:

1. A cultivation bottle for mycelium of Inonotus obliquus, comprising:
a bottle having a hollow shape with an opening; and
a bottle cap configured to cover the opening of the bottle, and comprising:
an annular cap, defining a space formed inside;
a press cap having a bottom part and a press part, and disposed in the space; and
at least one elastic member disposed under the press part and in the space, to enable the press part and the bottom part to enclose the space.

2. The cultivation bottle according to claim 1, further comprising a matrix placed within the bottle, wherein the matrix comprises an agar plate, sawdust distributed uniformly in the agar plate, and Inonotus obliquus strain seeded into the agar plate.

3. The cultivation bottle according to claim 1, wherein the annular cap, the press part and the bottom part are configured to define the space for containing culture liquid.

4. The cultivation bottle according to claim 1, wherein the annular cap comprises an annular part having an inner periphery and a plurality of ventilation holes cut therethrough, and an annular wall extended from the inner periphery of the annular part into the bottle and configured to define the space.

5. The cultivation bottle according to claim 4, wherein the annular cap comprises a rotatable part which is disposed on the annular part and configured to rotate to enclose a part or all of the ventilation holes.

6. The cultivation bottle according to claim 4, wherein the annular part has an outer periphery configured to cover the opening of the bottle, the annular wall has a step structure, and the elastic member is pressed between the step structure and the press part.

7. The cultivation bottle according to claim 4, wherein the annular wall has a structure formed on a lower periphery thereof and gradually-extended outwardly, the bottom part of the press cap has a soft rubber disposed on a periphery edge thereof, so as to prevent the culture liquid within the space from leaking when the press part is not pressed.

8. The cultivation bottle according to claim 1, wherein the press cap has a rod disposed between the press part and the bottom part within the space, and configured to press the bottom part to form a slit between the bottom part and a lower periphery of the annular cap while the press part is pressed.

9. The cultivation bottle according to claim 1, wherein the press part has a liquid inlet for inputting the culture liquid into the space.

10. A cultivation bottle for mycelium of Inonotus obliquus, comprising:
a bottle having a hollow shape with an opening;
a matrix placed within the bottle, wherein the matrix has an agar plate, sawdust distributed uniformly in the agar body, and Inonotus obliquus strain seeded into the agar body; and
a bottle cap configured to cover the opening of the bottle, and comprising:
an annular cap having a plurality of ventilation holes cut therethrough, and comprising an annular wall and a bottom part configured to define a space for containing culture liquid, wherein the bottom part has at least one gap enclosed by a rubber diaphragm; and
a press cap having a press part and configured to enclose the space;
wherein, when the press part of the press cap is pressed, compressed gas within the space forces the rubber diaphragm to open the gap of the bottom part, so that the culture liquid in the space is dropped into the matrix.
